(19) United States
(12) Patent Application Publication
(10) Pub. No.: US 2023/0361326 A1
(43) Pub. Date: Nov. 9, 2023

(54) DEVICE FOR DETERMINING THE HYDROGEN CONCENTRATION OF AN EXHAUST GAS IN AN EXHAUST GAS LINE OF A FUEL CELL SYSTEM, AND FUEL CELL SYSTEM

(71) Applicant: Robert Bosch GmbH, Stuttgart (DE)

(72) Inventors: Timo Bosch, Stuttgart (DE); Tobias Falkenau, Herrenberg (DE)

(21) Appl. No.: 18/246,375

(22) PCT Filed: Sep. 22, 2021
(86) PCT No.: PCT/EP2021/076036

§ 371 Date: Mar. 23, 2023

(30) Foreign Application Priority Data
Sep. 25, 2020 (DE) 10 2020 212 110.9

(51) Int. Cl. H01M 8/0444; G01N 33/00

(52) U.S. Cl. CPC H01M 8/04462; G01N 33/005

(57) ABSTRACT

The invention relates to a device (1) for determining the hydrogen concentration of a fluid in an exhaust gas line (12) of a fuel cell system (100), comprising a sensor (14) which is arranged in a tube section (2), wherein said tube section has an inflow opening (4) and an outflow opening (6). A purge line (40) opens into the tube section (2) between the inflow opening (4) and the H2 sensor (14). A mixing element (8) mixes an exhaust gas flowing through the inflow opening (4) such that different components of the exhaust gas are distributed homogeneously.

### BACKGROUND OF THE INVENTION

The invention relates to a device for determining the hydrogen concentration of an exhaust gas in an exhaust gas line of a fuel cell system, the device comprising a sensor arranged in a tube section, wherein the tube section has an inflow opening and an outflow opening.

The invention also relates to a fuel cell system comprising at least one fuel cell stack, an air path, wherein air from the environment reaches the fuel cell via the air path, an exhaust gas line, a fuel line, wherein fuel is transported to the fuel cell stack via the fuel line, and a circulation line, wherein the circulation line comprises a purge line.

Hydrogen-based fuel cells are considered to be the mobility concept of the future, because they emit only water as exhaust gas and allow fast refueling times. Fuel cells are usually assembled into a fuel cell stack. The fuel cell stacks use oxygen, mostly obtained from simple air from the environment, and fuel, mostly hydrogen, for the chemical reaction.

It is known that nitrogen reaches the cathode side of the fuel cell stack via the air mass flow, which is supplied to the fuel cell stack via the air path. Part of this nitrogen diffuses across the membrane of the fuel cell stack to the anode side and displaces the hydrogen on the anode side, so that the normal reactions are inhibited. To reduce the proportion of nitrogen on the anode side, a valve with a flushing line can lead from the anode side, or from the circulation line, to the exhaust gas line of the fuel cell to discharge anode gas with a high proportion of nitrogen to the environment via the exhaust gas line. To check the proportion of hydrogen in the exhaust gas line, a hydrogen sensor is arranged in the exhaust gas line.

### SUMMARY OF THE INVENTION

The device according to the invention for determining the hydrogen concentration of an exhaust gas in an exhaust gas line of a fuel cell system and the fuel cell system having the features according to the invention has the advantage that the hydrogen content in the exhaust gas line can be determined with greater accuracy. This is important so that, if necessary, measures can be taken to avoid an excessively high concentration of hydrogen and thus an explosive mixture.

By means of the mixing element according to the invention, a fluid flowing through the inflow opening is mixed by a change in the flow ratios, so that different components within the exhaust gas have a homogeneous distribution.

Without the device according to the invention, there is a risk that the sensor will detect a too low or too high concentration of hydrogen in the exhaust gas, because the purge gas has not evenly distributed in the exhaust gas line and, potentially, a too low or too high concentration of hydrogen that does not match the mean concentration value will be present locally at the position of the sensor.

Advantages in relation to the design space result from the devices according to the invention, because a shorter line section is required until a uniform mixing of hydrogen and the further exhaust gases in the exhaust gas line results. Consequently, the exhaust gas line can be shorter.

If a too high concentration of hydrogen is detected, there is the possibility to selectively increase the air mass flow in the exhaust gas line, which can potentially be fed directly from the air path into the exhaust gas line via a bypass terminal. Another option is to stop or reduce the supply of anode gas. Another option is to catalytically burn the hydrogen.

Advantageous configurations and further developments of the device according to the invention for determining the hydrogen concentration of an exhaust gas in an exhaust gas line of a fuel cell system and the fuel cell system are specified in the dependent claims.

It is advantageous when the device comprises a terminal for a purge line, wherein a purge gas of the purge line is directed into the tube section via the terminal. The shape and position of the mixing element can be selected by a defined inflow location of the purge gas such that the sensor is as homogeneously mixed as possible with the purge gas from the purge gas line and the exhaust gas from the exhaust gas line.

It is advantageous here when the terminal for the purge line is arranged between the inflow opening and the mixing element. However, it is also possible for the inflow opening to be placed at the height of the mixing element, so that the exhaust gas from the exhaust gas line has already been altered in its flow behavior when the purge gas enters.

An advantageous structure for a mixing element can be formed by a spiral structure, a lattice structure or a cascade of diverting plates.

A particular advantage arises when the purge gas does not flow selectively via the terminal, but rather at several introduction points into the tube section, because in this way an initial distribution of the purge gas is already ensured. A radial distribution of the introduction points over an outer wall of the tube section is advantageous here.

Due to the even higher flexibility and thus the better mixing, it is advantageous when the introduction points are arranged on the mixing element so that purge gas flows in the axial and/or radial direction into the tube section.

Depending on the local flow conditions, the installation element makes it possible to fix the sensor to an outer wall of the tube section or to the installation element as needed.

### BRIEF DESCRIPTION OF THE DRAWINGS

The device according to the invention and the fuel cell system according to the invention are explained in more detail in the following with reference to drawings. Schematically, the figures show:

FIG. 1 a schematic topology of a fuel cell system according to a first embodiment example of the invention,

FIG. 2 a device for determining the hydrogen concentration of a fluid in an exhaust gas line of a fuel cell system in a schematic illustration,

FIG. 3 a schematic topology of a fuel cell system according to a second embodiment example of the invention, and

FIG. 4 devices for determining the hydrogen concentration of a fluid in an exhaust gas line of a fuel cell system with different installation elements in a schematic illustration, and

FIG. 5 a device for determining the hydrogen concentration of a fluid in an exhaust gas line of a fuel cell system with an installation element configured as a tube in a schematic illustration, and

FIG. 6 a further device for determining the hydrogen concentration of a fluid in an exhaust gas line of a fuel cell system in a schematic illustration.

### DETAILED DESCRIPTION

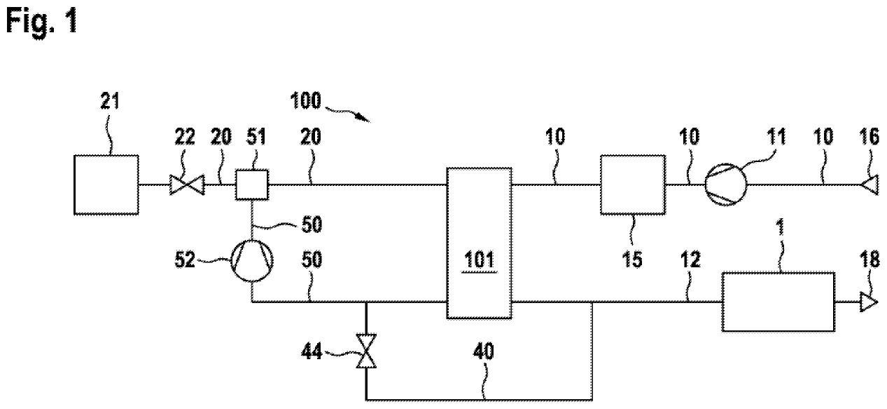

FIG. 1 shows a schematic topology of a fuel cell system 100 according to a first embodiment example with at least one fuel cell stack 101. The at least one fuel cell stack 101 comprises an air path 10, an exhaust gas line 12 and a fuel line 20. The at least one fuel cell stack 101 can be used for mobile applications with high power requirement, for example in trucks, or for stationary applications, for example in generators.

The air path 10 serves as an air supply line for supplying air from the environment to the fuel cell stack 101 via an inflow 16. Components needed for the operation of the fuel cell stack 101 are arranged in the air path 10. An air compressor 11 and/or compressor 11, which compresses and/or draws in the air in accordance with the respective operating conditions of the fuel cell stack 101, is arranged in the air path 10. A humidifier 15 which increases the water content of the air in the air path 10 can be arranged downstream of the air compressor 11 and/or compressor 11.

Further components, such as a filter and/or a heat exchanger and/or valves, can be provided in the air path 10 as well. Air containing oxygen is made available to the fuel cell stack 101 via the air path 10.

The fuel cell system 100 also comprises an exhaust gas line 12 in which water and other components of the air from the air path 10 are transported into the environment via an outflow 18 after passing through the fuel cell stack 101. The exhaust gas of exhaust gas line 12 can also contain hydrogen (H2), because portions of the hydrogen can diffuse through the membrane of the fuel cell stack 101.

The fuel cell system 100 can moreover comprise a cooling circuit configured to cool the fuel cell stack 101. The cooling circuit is not shown in FIG. 1 because it is not part of the invention.

A high pressure tank 21 and a shut-off valve 22 are arranged in the inflow of fuel line 20. Additional components can be arranged in the fuel line 20 to supply fuel to the fuel cell stack 101 as needed.

To always adequately supply the fuel cell stack 101 with fuel, there is a need for an over-stoichiometric metering of fuel via the fuel line 20. The excess fuel, and also certain amounts of water and nitrogen that diffuse through the cell membranes to the anode side, are recirculated in a recirculation line 50 and mixed with the metered fuel from the fuel line 20.

Various components, such as a jet pump 51 operated with the metered fuel or a blower 52, can be installed to drive the recirculation circuit 50. A combination of jet pump 51 and blower 52 are possible as well.

Because the amount of water and nitrogen increases more and more over time, the recirculation circuit 50 must be flushed periodically so that the performance of the fuel cell stack 101 does not decrease due to an excessive concentration of nitrogen in the fuel line 20.

A purge line 40 is arranged between the circulation line 50 and the exhaust gas line 12 so that the gas mixture can flow from the circulation line 50 into the exhaust gas line 12.

A purge valve 44 which can open and close the terminal between the circulation line 50 and the exhaust gas line 12 can be arranged in the purge line 40. The purge valve 44 is typically opened for a short period of time, so that the gas mixture is fed into the exhaust gas line 12 via the purge line 40.

According to one embodiment of the invention, a device 1 for determining the hydrogen concentration is arranged in the exhaust gas line 12.

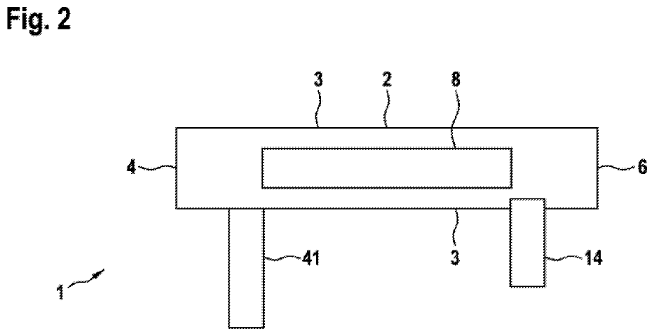

FIG. 2 shows a device 1 for determining the hydrogen concentration in a schematic illustration. The device 1 is formed by a tube section 2 comprising a sensor 14, which can measure the hydrogen concentration in a fluid. The tube section 2 comprises an inflow opening 4 and an outflow opening 6. Furthermore, a mixing element 8 is arranged in the tube section 2, which mixes the fluid that has passed from the exhaust gas line 12 through the inflow opening 4 so that different components within the exhaust gas have a homogeneous distribution.

In a further embodiment of the invention, the tube section 2 can comprise a terminal 41 for the purge line 40.

The terminal 41 for the purge line 40 is arranged between the inflow opening 4 and the mixing element 8, so that, when measuring, the sensor 14 measures the hydrogen concentration from both the exhaust gas line 12 and the purge line 40.

FIG. 1 shows a fuel cell system 100 comprising a device without a terminal 41, in which the purge line 40 opens into the exhaust gas line 12 in front of the device 1 in the direction of flow.

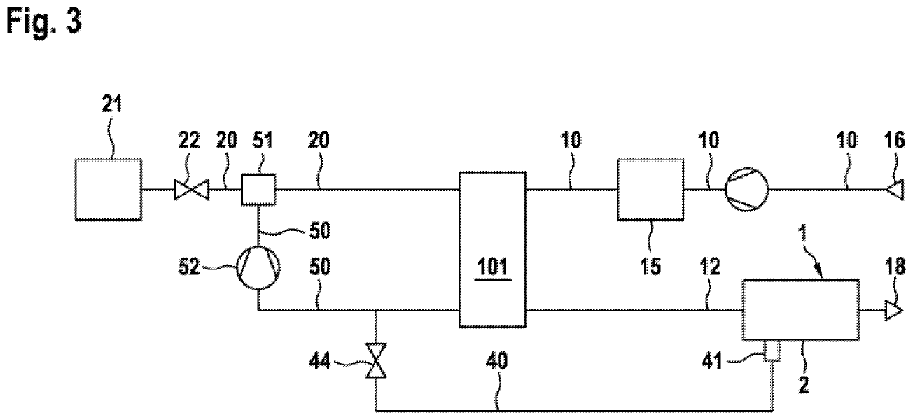

FIG. 3 shows a fuel cell system 100 comprising a device 1 with a terminal 41. Here the purge line 40 is connected to the terminal 41, so that the purge gas can flow directly from the purge line 40 via the terminal 41 into the device.

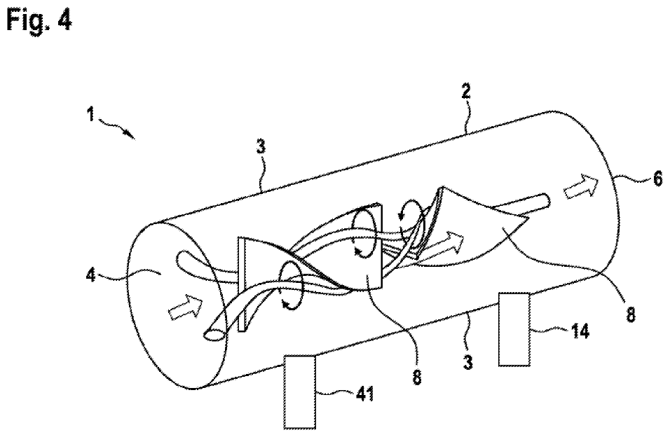

FIG. 4 shows a device 1 with a mixing element 8, which is formed from twisted rectangular plates. The turbulent or laminar flow profiles are destroyed by the mixing element, so that a larger mixing of the individual fluid particles within a flow cross-section in the tube section occurs. In the figure, the turbulence of the fluid is indicated by arrows.

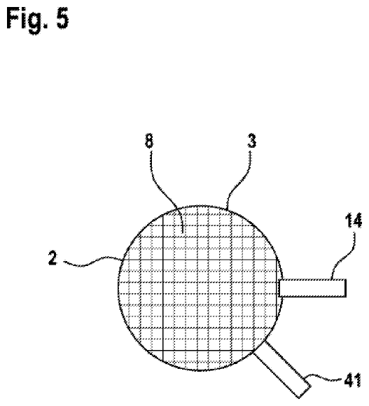

FIG. 5 shows a device 1 in which the mixing element 8 is formed by a flow lattice. In further embodiments of the invention, the mixing element 8 can be formed by a spiral structure or cascade of diverting plates.

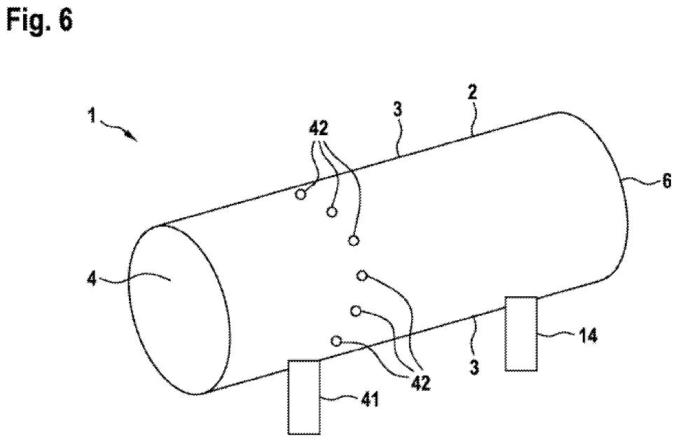

FIG. 6 shows an embodiment of the invention, in which the purge gas does not flow selectively through the terminal 41 but rather at several introduction points 42 into the tube section 2. In this case, the introduction points 42 can be radially distributed over an outer wall of the tube section 2. It is also possible for the introduction points 42 to be arranged on the mixing element 8 so that purge gas flows in the axial and/or radial direction into the tube section 2.

The sensor 14 can be attached to an outer wall 3 of the tube section 2 or to the mixing element 8.

1. A device (1) for determining the hydrogen concentration of an exhaust gas in an exhaust gas line (12) of a fuel cell system (100), the device comprising a sensor (14) arranged in a tube section (2), wherein the tube section (2) has an inflow opening (4) and an outflow opening (6), the device also comprising a mixing element (8) configured to mix a fluid which flows through the inflow opening (4) such that different components of the exhaust gas are distributed homogeneously.

2. The device according to claim 1, characterized in that the tube section (2) comprises a terminal (41) for a purge line (40), wherein a purge gas of the purge line (40) is fed into the tube section (2) via the terminal (41).

3. The device (1) according to claim 2, characterized in that the terminal (41) for the purge line (40) is arranged between the inflow opening (4) and the mixing element (8).

4. The device (1) according to claim 1, characterized in that the mixing element (8) is formed by a spiral structure, a lattice structure, or a cascade of diverting plates.

5. The device (1) according to claim 2, characterized in that the purge gas does not flow through the terminal (41) selectively but rather at several introduction points (42) into the tube section (2).

6. The device (1) according to claim 5, characterized in that the introduction points (42) are radially distributed over an outer wall (3) of the tube section (2).

7. The device (1) according to claim 5, characterized in that the introduction points (42) are arranged on the mixing element (8) so that purge gas flows in the axial and/or radial direction into the tube section (2).

8. The device (1) according to claim 1, characterized in that the sensor (14) is fixed to an outer wall (3) of the tube section (2) or to the mixing element.

9. A fuel cell system (100) comprising at least one fuel cell stack (101), an air path (10), wherein air from the environment reaches the fuel cell via the air path (10), an exhaust gas line (12), a fuel line (20), wherein fuel is transported to the fuel cell stack (101) via the fuel line (20), and a circulation line (50), wherein the circulation line (50) comprises a purge line (40), characterized in that a device (1) according to claim 1 is arranged in the exhaust gas line (12).

10. The fuel cell system (100) according to claim 9, characterized in that the purge line (40) is connected to the terminal (41) of the device (1).

11. The device (1) according to claim 1, characterized in that the mixing element (8) is formed by a spiral structure.

12. The device (1) according to claim 1, characterized in that the mixing element (8) is formed by a lattice structure.

13. The device (1) according to claim 1, characterized in that the mixing element (8) is formed by a cascade of diverting plates.

14. The device (1) according to claim 1, characterized in that the sensor (14) is fixed to an outer wall (3) of the tube section (2).

15. The device (1) according to claim 1, characterized in that the sensor (14) is fixed to the mixing element.
